(19) United States
(12) Patent Application Publication
(10) Pub. No.: US 2023/0112275 A1
(43) Pub. Date: Apr. 13, 2023

(54) INFERRING DEVICE, INFERRING METHOD, STRUCTURAL FORMULA, AND NON-TRANSITORY COMPUTER READABLE MEDIUM

(71) Applicant: Preferred Networks, Inc., Tokyo-to (JP)

(72) Inventors: Ryuichiro ISHITANI, Tokyo-to (JP); Motoki ABE, Tokyo-to (JP)

(73) Assignee: Preferred Networks, Inc., Tokyo (JP)

(21) Appl. No.: 18/076,636

(22) Filed: Dec. 7, 2022

Related U.S. Application Data
(63) Continuation of application No. PCT/JP2021/021850, filed on Jun. 9, 2021.

(30) Foreign Application Priority Data
Jun. 9, 2020 (JP) 2020-100403

(51) Int. Cl. G06F 17/11

(52) U.S. Cl. CPC G06F 17/11

(57) ABSTRACT

An inferring device includes one or more memories and one or more processors. The one or more processors are configured to acquire a plurality of latent variables; generate a plurality of structural formulas by inputting the plurality of latent variables, respectively, in a model; and calculate a plurality of scores by evaluating the plurality of structural formulas, respectively. The one or more processors execute processing of the acquisition of the plurality of latent variables, the generation of the plurality of structural formulas, and the calculation of the plurality of scores, at least two times or more. The one or more processors acquire, based on the acquired plurality of latent variables and the calculated plurality of scores, the plurality of latent variables in any of the execution of at least second time or thereafter.

### CROSS REFERENCE TO RELATED APPLICATIONS

This application is continuation application of International Application No. JP2021/021850, filed on Jun. 9, 2021, which claims priority to Japanese Patent Application No. 2020-100403, filed on Jun. 9, 2020, the entire contents of which are incorporated herein by reference.

### FIELD

The present disclosure relates to an inferring device, an inferring method, a structural formula, and a non-transitory computer readable medium.

### BACKGROUND

Studies regarding generation of structural formulas by using an algorithm of machine learning and the like, have been performed actively. A model generated by these studies is one that outputs, when a mathematical symbol such as a latent variable is given thereto, a structural formula corresponding to the mathematical symbol. This model sometimes outputs a plurality of structural formulas, and it sometimes outputs one or a plurality of structural formulas stochastically from a plurality of candidates.

By these methods, the generation of structural formula itself becomes possible, but it is difficult to automatically generate a structural formula of a compound having a preferable chemical property. In order to generate the structural formula of the compound having the preferable chemical property, it is required to continuously and automatically generate a plurality of structural formulas, and to keep performing the generation until when the structural formula of the compound having the preferable chemical property can be obtained, which takes quite a long time.

### BRIEF DESCRIPTION OF DRAWINGS

FIG. 1 is a block diagram schematically illustrating an inferring device according to one embodiment;

FIG. 2 is a flow chart illustrating processing of the inferring device according to the embodiment;

FIG. 3 is a block diagram schematically illustrating implementation of at least a part of the inferring device according to the embodiment;

FIG. 4 is a flow chart illustrating processing of the inferring device according to the embodiment; and

FIG. 5 is an example of a hardware implement of one embodiment.

### DETAILED DESCRIPTION

According to some embodiments, an inferring device includes one or more memories and one or more processors. The one or more processors are configured to acquire a plurality of latent variables; generate a plurality of structural formulas by inputting the plurality of latent variables, respectively, in a model; and calculate a plurality of scores by evaluating the plurality of structural formulas, respectively. The one or more processors execute processing of the acquisition of the plurality of latent variables, the generation of the plurality of structural formulas, and the calculation of the plurality of scores, at least two times or more. The one or more processors acquire, based on the acquired plurality of latent variables and the calculated plurality of scores, the plurality of latent variables in any of the execution of at least second time or thereafter.

Hereinafter, embodiments of the present invention will be explained while referring to the drawings. The explanation of the drawings and the embodiments is presented as an example, and does not limit the present invention.

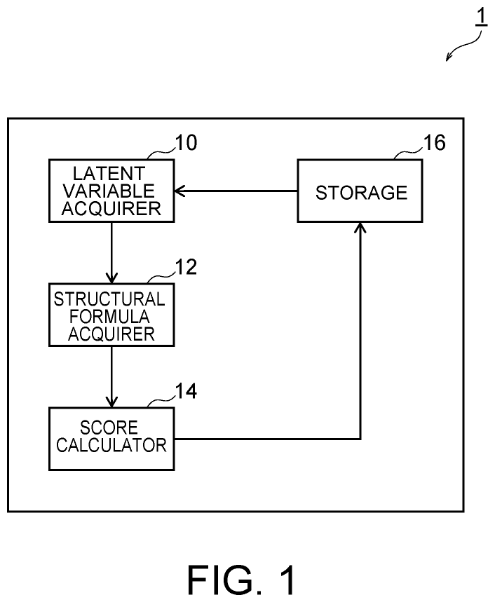

FIG. 1 is a block diagram schematically illustrating an inferring device according to the present embodiment. An inferring device 1 includes a latent variable acquirer 10, a structural formula acquirer 12, a score calculator 14, and a storage 16.

The latent variable acquirer 10 acquires a latent variable that is used in the structural formula acquirer 12. This latent variable may be coordinates indicating one point in a multidimensional latent space. Specifically, in the following explanation, the term of latent variable is sometimes used as a meaning of the coordinates indicating one point in the latent space. This latent variable includes, for example, a hidden vector, a latent vector, and the like.

As will be described later, the latent variable acquirer 10 acquires a latent variable based on a metaheuristic algorithm. In the present disclosure, the latent variable may include a scalar, or a concept including a matrix being a vector of two dimensions or more, and further, it may also be a concept including a tensor as a result of further generalizing these.

The structural formula acquirer 12 acquires a structural formula based on the latent variable acquired by the latent variable acquirer 10. The structural formula expresses a structure of a compound such as a molecule or crystal, two-dimensionally in a pseudo manner, for example. The structural formula acquirer 12 may infer and acquire a structural formula by one method, and in addition to that, it may also infer and acquire a structural formula by a plurality of methods. Further, it is also possible to execute the inference of structural formula by a method of performing complementary inference. For instance, a structure of a compound can be expressed by a graph.

The score calculator 14 calculates a score with respect to the structural formula acquired by the structural formula acquirer 12. The score is determined based on a chemical property which the compound expressed by the structural formula has, for example. For instance, the score calculator 14 may calculate a plurality of scores from a plurality of properties, and then calculate a score indicated by one scalar from these plurality of scores. The score calculator 14 may establish a link between the latent variable and the score to store them in the storage 16.

The storage 16 may store information of the latent variable acquired by the latent variable acquirer 10 and information of the score. In addition to that, when the inferring device 1 is concretely realized by hardware based on processing of software, the storage 16 may store a program and the like (including OS and the like) which execute the processing of the software. The storage 16 may be provided in the inferring device 1, and it may also be provided outside the inferring device 1. For example, the storage 16 may be provided in a server via a network such as the Internet, or on a cloud.

The inferring device 1 may include, other that the above, an input interface that inputs data, and an output interface that outputs a result to the outside.

Next, regarding each configuration, one example of processing will be described.

The structural formula acquirer 12 may acquire a structural formula from a latent variable based on a method such as, for example, Junction Tree VAE (Variational Autoencoder) or AAE (Adversarial Autoencoder). For example, a latent variable is input in a model generated based on these methods, thereby acquiring a structural formula. For example, in a case of a model of Junction Tree VAE, a 56-dimensional latent variable is input to acquire a structural formula, and in a case of a model of AAE, a 32-dimensional latent variable is input to acquire a structural formula. A latent variable is input in an intermediate layer of a model generated by an autoencoder, for example, a layer of a decoder, and the model outputs a structural formula based on a feature amount indicated by the latent variable.

The score calculator 14 measures a structural feature of the structural formula acquired by the structural formula acquirer 12, as one of the scores described above, and acquires a chemical property from this structural feature. The acquisition of the chemical property is executed by a docking simulation, for example. The score calculator 14 calculates the score based on this chemical property, for example. For instance, the score may be calculated based on at least one of a position of a certain compound and a potential function, obtained by the docking simulation. The position of the compound indicates that at which position of certain protein the compound bonds, for example. More concretely, the score calculator 14 calculates a score based on various methods, and based on this score, the latent variable acquirer 10 acquires a latent variable by using various optimization methods, as will be described later.

The score calculator 14 may take not only the position of the compound but also a direction of the compound into consideration. The structural formula itself is one having one-dimensional or two-dimensional structure, but actually, the compound has a three-dimensional structure. Accordingly, for the compound expressed by the structural formula, not only its docking position but also a docking direction and internal coordinates become important elements as well. For this reason, the score calculator 14 may also set the docking direction as an evaluation target. For example, regarding a compound used as a medical agent, information indicating that at which degree of affinity the compound bonds to protein, becomes important. In such a case, by performing evaluation with a score while considering at which conformation the compound bonds to protein, together with the position of the compound, it becomes possible to improve accuracy of the score.

For example, the evaluation of the docking is executed by judging that at which position and a posture (including a conformation) the above-described compound bonds to protein, and calculating the degree of chemical energy when the bonding occurs. The compound used for the algorithm of the docking is a compound generated by the structural formula acquirer 12 based on the algorithm of Junction Tree VAE, AAE that generates various structural formulas, and the like. When the compound generated by the various algorithms is bonded to protein, it can be evaluated that the lower the energy, the higher the possibility of actual bonding. Accordingly, it is possible to use magnitude (high or low) of energy taken for the bonding, as an evaluation value.

Specifically, as an example, the inferring device 1 searches for a “compound” having a condition such that it properly bonds to protein or it easily bonds to protein. More specifically, the interring device 1 uses the docking algorithm to execute the evaluation, and calculates a score indicating the easiness of bonding between the compound acquired from the latent variable and protein while taking a position and a posture at which the compound and protein bond into consideration. Subsequently, the inferring device 1 searches for, based on this score, the compound capable of properly bonding to protein.

The score calculator 14 evaluates the compound acquired by the structural formula acquirer 12 based on a chemical property and the like which the compound has or may have, to thereby calculate a score. This score may be output so as to be utilized for multiple purposes. Specifically, the score may be calculated based on a properly-defined calculation method, based on a situation and the like under which the compound is used.

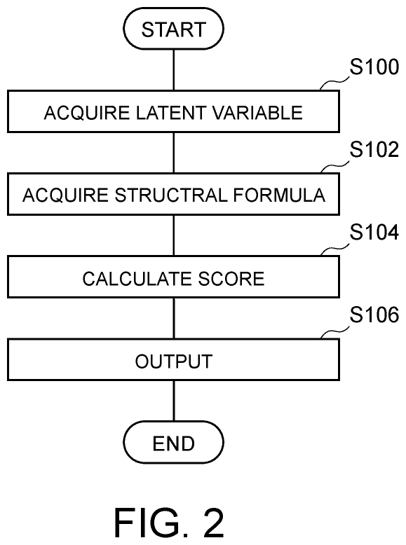

FIG. 2 is an example of a flow chart of the present embodiment.

First, the latent variable acquirer 10 acquires a latent variable based on a metaheuristic algorithm (S100), as will be described later. The latent variable acquirer 10 acquires, based on an already-obtained score, a latent variable such as one capable of acquiring a further desirable score.

Next, the structural formula acquirer 12 acquires a structural formula from the acquired latent variable (S102). As described above, the structural formula acquirer 12 may acquire a structural formula from one model, or it may also acquire a structural formula from a plurality of models. When acquiring a structural formula from a plurality of models, the latent variable acquirer 10 acquires, in S100, a latent variable which is proper to be input in a model to be used. This generation of structural formula can employ various algorithms, as described above. The latent variable acquirer 10 acquires the latent variable suitable for the algorithm used in the structural formula acquirer 12.

Next, the score calculator 14 calculates a score with respect to the acquired structural formula (S104). The score calculator 14 calculates a docking score based on the above-described evaluation method.

Next, the inferring device 1 properly outputs information (S106). For example, the inferring device 1 may convert data into visual information or the like being information capable of being perceived by a user, to thereby acquire a structural formula and a score. For instance, the inferring device 1 may output the latent variable and the score to the storage 16. Other than the above, the inferring device 1 may output proper information out of the latent variable, the structural formula, and the score obtained above, to a proper place via the output interface.

The present embodiment can be applied to a pharmaceutical field, for example. When the present embodiment is applied to pharmacy, for instance, the score may be adjusted in the score calculator 14 while setting a compound having toxicity with respect to humans or animals, or the like to one having an avoidance structure. It is possible to configure such that the latent variable acquirer 10 performs a later-described optimization based on this score, to thereby acquire a latent variable capable of eventually avoiding the avoidance structure.

As the chemical property used for the score calculation, it is possible to set performance as cosmetics or high-performance materials, other than the activity of medicines, pesticides, and the like described above, to the score. The chemical property is not limited to these, and one that requires inference regarding a molecular structure can be properly applied.

Next, more concrete contents regarding the way of acquiring the latent variable, will be described.

The latent variable acquirer 10 acquires a plurality of latent variables based on the metaheuristic algorithm.

The structural formula acquirer 12 acquires a plurality of structural formulas based on the latent variables acquired by the latent variable acquirer 10 based on the metaheuristic algorithm.

The score calculator 14 calculates scores with respect to the plurality of structural formulas acquired by the structural formula acquirer 12 based on the metaheuristic algorithm. The score calculator 14 performs an arithmetic operation, in a parallel manner, on the plurality of structural formulas acquired by the structural formula acquirer 12. Furthermore, the score calculator 14 performs an arithmetic operation, in a parallel manner, on docking scores regarding various conformations, with respect to a plurality of compounds, respectively.

Specifically, the score calculator 14 performs the arithmetic operation, in a parallel manner, on the plurality of docking scores regarding the plurality of structural formulas, respectively, generated by the metaheuristic algorithm. As described above, the inference of the compound according to the present embodiment is efficiently executed with respect to a plurality of compounds at the same timing.

The pieces of processing in the latent variable acquirer 10, the structural formula acquirer 12, and the score calculator 14, may be realized as parallel processing. The parallel processing may be executed by using a GPU (Graphics Processing Unit) or the like, for example. The parallel processing does not always have to be executed by the GPU, and it may also be executed by using a general-purpose processing circuit such as a CPU (Central Processing Unit), a dedicated circuit such as an ASIC (Application Specific Integrated Circuit), or a programmable processing circuit such as a FPGA (Field Programmable Gate Array).

As an example of the above-described metaheuristic algorithm, it is possible to use a Particle Swarm Optimization (PSO). Other than the PSO, it is possible to use various algorithms such as, for example, an Artificial Bee Colony (ABC) method, a Metropolis method/Monte Carlo method such as a Simulated Annealing (SA) method, a Hill Climbing (HC) method and an application method thereof which does not use a derivative, an Ant Colony Optimization (ACO) method, a Harmony Search (HS), a Cuckoo Search (CS), a Spiral Optimization method, a Firefly Algorithm, an Evolution Strategy (ES) such as Genetic Algorithm (GA)/Immune Algorithm/Covariance Matrix Adaptation Evolution Strategy (CMA-ES), and an Amoeba Method/Nelder-Mead Method. The arithmetic efficiency thereof can be improved by realizing the parallel processing with the use of a plurality of arithmetic cores.

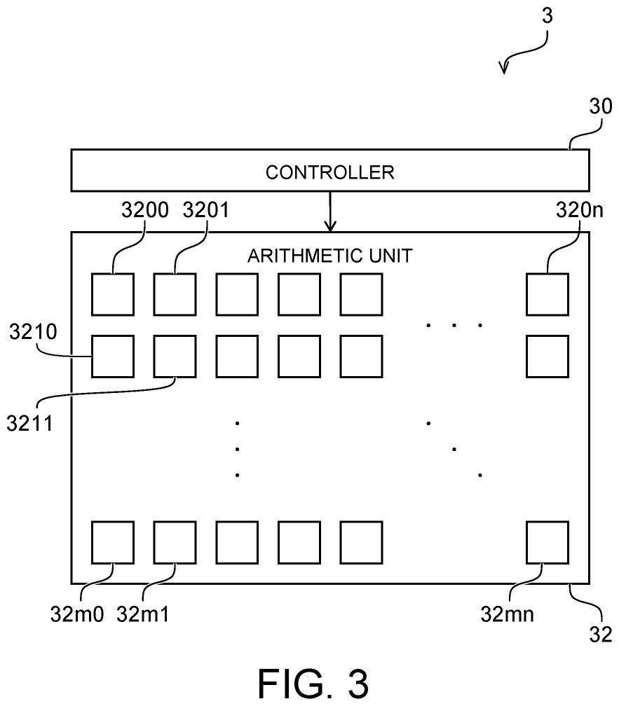

FIG. 3 is a schematic view illustrating an arithmetic device being a part of implementation of the inferring device 1 according to the present embodiment. An arithmetic device 3 includes a controller 30 and an arithmetic unit 32. Further, the controller 30 and the arithmetic unit 32 may have a not-illustrated storage, respectively, or they may have a memory region which can be used in a shared manner. Further, it is set that a communication path such as a bus is properly provided.

The controller 30 controls arithmetic processing. This controller 30 is formed by including an electronic circuit, for example.

The arithmetic unit 32 executes arithmetic processing based on a request from the controller 30. As illustrated in the drawing, the arithmetic unit 32 may have a plurality of arithmetic cores 3200, 3201, . . . , 320n, 3210, 3211, . . . , 32m0, 32m1, . . . , 32mn. As an example, the arithmetic unit 32 is configured to have m×n arithmetic cores arranged in parallel in a grid form, but the configuration is not limited to this.

These arithmetic cores may be divided into a plurality of groups in which a plurality of threads are executed in a synchronized manner. Regarding a plurality of groups that belong to the same group, predetermined plural groups may be further synchronized with each other through atomic processing. Furthermore, it is possible to configure such that all the groups or arithmetic cores execute the arithmetic operation in a synchronized manner. As described above, the multithreaded processing may be executed in a properly synchronized manner in the arithmetic unit 32.

When the plurality of arithmetic cores are operated in a synchronized manner, similar arithmetic processing can be executed by the plurality of arithmetic cores. For example, when executing the metaheuristic algorithm, by giving pieces of information to which different parameters are given, to the plurality of arithmetic cores, respectively, it becomes possible to output different arithmetic results.

It is possible to provide a memory such as a cache which is used in a shared manner by at least a plurality of configurations out of a part or all of the arithmetic cores, and the controller. The memory such as the cache may be shared by every predetermined number of arithmetic cores. For example, there may exist a memory region capable of being accessed in a predetermined arithmetic core, or there may also exist a memory region capable of being accessed from the controller (host side) as well. When reading the cache, it is possible to execute control capable of suppressing a bank conflict.

For example, the latent variable acquirer 10, the structural formula acquirer 12, and the score calculator 14 may be implemented in each of these arithmetic cores. These components may be operated in a synchronized manner among a part or all of the predetermined arithmetic cores, as described above.

The pieces of processing of the plurality of arithmetic cores belonging to the arithmetic unit 32 are executed by the controller 30. In FIG. 3, one set of arithmetic cores is provided, as the arithmetic unit 32, with respect to the controller 30, but not limited to this. For example, the controller 30 can execute control of the arithmetic processing with respect to a plurality of different sets of arithmetic cores. Further, the controller 30 and the arithmetic unit 32 may be provided to the same accelerator or the like such as the GPU. As another example, the controller 30 may control the accelerator or the like from a host computer that is different from the arithmetic unit 32. In this case, it is possible to employ a configuration in which a controller of the host and a controller of a client such as the accelerator are provided in multiple stages. For example, in a case of the GPU, it is often the case where one thread is assigned to one arithmetic core to perform one arithmetic operation, but not limited to such a configuration, and it is also possible to use an arithmetic unit having a configuration in which the arithmetic operation can be performed in a parallel manner in one arithmetic core as well.

In any of the above cases, the accelerator or the like may be a heterogeneous or homogeneous multi-core processor, or a many-core processor.

The processing with respect to the plurality of arithmetic cores may be executed by a SIMD (Single Instruction, Multiple Data) instruction, a MIMD (Multiple Instruction, Multiple Data) instruction, or a MISD (Multiple Instruction, Single Data) instruction, and it may also be executed by being properly combined with processing of executing these instructions. Further, these arithmetic cores may be tightly coupled or loosely coupled. Furthermore, the arithmetic cores may also be implemented in a manner that the tightly-coupled arithmetic cores and the loosely-coupled arithmetic cores exist at the same time. For example, the arithmetic unit 32 may be realized as grid computing in which a plurality of GPUs are connected by various communication lines, and it may be formed by a plurality of arithmetic cores provided to the plurality of GPUs.

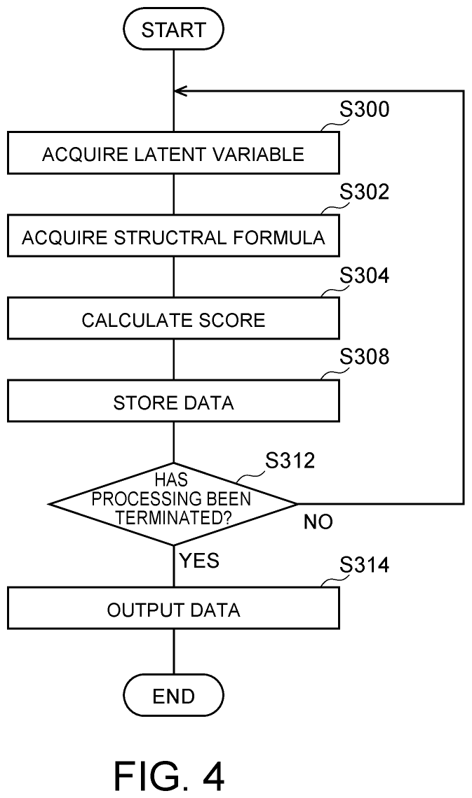

FIG. 4 is a flow chart illustrating processing of the inferring device 1 according to the present embodiment.

First, the latent variable acquirer 10 acquires a latent variable (S300). As described above, the latent variable acquirer 10 is implemented so that it is operated in the arithmetic core. The controller 30 controls the respective arithmetic cores of the arithmetic unit 32 so as to be able to acquire a plurality of latent variables in a parallel manner, based on the metaheuristic algorithm.

In a first arithmetic operation in the processing, it is possible to set a plurality of random numbers as latent variables, for example. Further, as another example, it is also possible to set a plurality of latent variables based on information of already-known structural formula and the like. For example, this information may be obtained from the linkage information between latent variables and scores stored in the storage 16 or the like, or it may be obtained in a manner that a database of latent variables and structural formulas, for example, is provided, and the information is extracted through collation of data of the database.

When the PSO is used for searching for the latent variable, with respect to each latent variable set as an initial value, each velocity vector with respect to the latent variable may be set by a random number. Further, with respect to the latent variable set as the initial value, an update of the latent variable by the initial velocity may be performed. For example, in the case of the PSO, the update of the latent variable and the velocity vector is performed as follows.

x=x+v (1)

v=wv+c₁r₁(\hat{x}ₚ−x)+c₂r₂(\hat{x}_{g}−x) (2)

Here, x indicates a latent variable, v indicates a velocity, w indicates an inertia constant, each of c₁ and c₂ indicates a proportion of particles directed to a good position within a group, each of r₁ and r₂ indicates a random number of [0,1], x\hat{ }ₚ indicates an optimum vector of focused particles in loops so far, and x\hat{ }_{g} indicates an optimum vector in the loops so far as the entire group.

In a second loop and thereafter, when the PSO method is used as the metaheuristic algorithm, for example, as the latent variable, a latent variable based on the structural formula acquired in the previous loop (graph data including a conformation) and the calculated score data, is generated by the control from the controller 30. For example, a docking score is used to extract the x\hat{ }ₚ and the x\hat{ }_{g} in the above formula, to thereby update the velocity v and the latent variable x. Note that the required data such as the relation between the score, and the x\hat{ }ₚ and the x\hat{ }_{g} in the PSO, for example, may be properly linked and stored in the storage 16 in each step.

The controller 30 sets parameters to the respective arithmetic cores so as to acquire a plurality of latent variables based on the docking score acquired until the previous loop, and the latent variable (structural formula) acquired at the present moment, as described above.

Further, also in the metaheuristic algorithm other than the PSO, the controller 30 similarly performs control so as to calculate respective latent variables in the respective arithmetic cores based on the evaluation value (docking score) acquired in the last loop or further previous loop and the latent variable. As described above, by using the plurality of arithmetic cores provided to the arithmetic unit 32, the search of latent variable is executed in a parallel manner.

Next, in each arithmetic core, the structural formula acquirer 12 acquires a structural formula based on the acquired latent variable (S302). The acquisition of structural formula with respect to each latent variable may be executed in a closed state in the arithmetic core that acquired the latent variable. By executing the acquisition as above, it becomes possible to acquire a plurality of structural formulas (including conformations) in a parallel manner.

Next, the score calculator 14 executes evaluation of the structural formula acquired by the structural formula acquirer 12 (S304). This evaluation is executed by calculating the docking score based on the information such as the three-dimensional structure, the atomic arrangement and conformation, and the internal coordinates with respect to the structural formula, as described above. This score calculation is processed by a parallel arithmetic operation, as will be described later.

Next, the inferring device 1 stores acquired data in the storage 16 (S308). The inferring device 1 stores, in the storage 16, at least data required for subsequent processing, and data which may be a final optimum solution. In this step, storage of data other than the above is also allowed.

Next, the inferring device 1 judges whether or not the optimization has been terminated, namely, the structural formula has been inferred as proper one (S312). This judgment is performed based on conditions such that, for example, the evaluation value has become lower than a predetermined numeric value, the number of times of repetition of loop has reached a predetermined number, or a predetermined number of the structural formulas whose evaluation values are lower than the predetermined numeric value have been searched. In a case where the higher the evaluation value, the better, a case where the evaluation value is greater than the predetermined numeric value, not the case where the evaluation value is lower than the predetermined numeric value, is set to the termination condition, as a matter of course. These conditions are cited as examples, and the processing may be terminated based on a condition other than these conditions.

When the processing has not been terminated (S312: NO), the processing from S300 is repeated.

When the processing has been terminated (S312: YES), the inferring device 1 outputs data, and terminates the processing (S314).

The above is the explanation of the flow of the entire processing in the inferring device 1. Hereinafter, the parallel processing of the docking score calculation will be described in more detail, by citing a case of using the Monte Carlo method and a BFGS method, as an example. Note that this explanation does not limit the contents of the invention of the present disclosure, and it is only required to employ an embodiment capable of performing processing from a plurality of compounds in a parallel manner.

These methods are indicated as examples, and it is only required to employ a method of properly calculating an optimum docking score that is not a local solution with respect to each structural formula. For instance, when using the Monte Carlo method and the BFGS method being one kind of a quasi-Newton method, a global search is performed by the Monte Carlo method, and a locally optimum solution with respect to a state selected by the Monte Carlo method (for example, coordinate values, conformation, and so on, for example) is searched by the BFGS method. Although a large temporal cost is required if such a method is successively performed, by performing the arithmetic operation of determining the local solution through the parallel arithmetic operation, the temporal cost is reduced.

Note that the BFGS method of using a docking score gradient is used as an example, but a method which does not use the gradient may also be employed. Further, in the following explanation, the Monte Carlo method is successively performed, but it is possible to execute the search in a parallel manner, namely, it is possible to execute a plurality of selections to be the reference of the BFGS method in a parallel manner.

When a compound and its three-dimensional structure are set to x, and a function that determines an evaluation value, namely, a function of determining a docking score from coordinate values of the compound (which may be atomic coordinate values or internal coordinate values, for example) is set to f(x), the gradient is calculated by the following formula. The f(x) is a scalar function in which an input is set to a vector.

∇f(x) (3)

Here, ∇ indicates differentiation of determining the gradient with respect to the vector x.

Bₖpₖ=−∇f(xₖ) (4)

Bₖ is set to an approximate matrix of Hessian matrix, and a search direction pₖ is calculated based on the formula (4). Here, k indicates an iteration number of loop.

pₖ=−Bₖ⁻¹∇f(x) (5)

When the formula (4) is modified, it can be written as the formula (5). Specifically, with respect to the coordinate values to be the reference in the loop, the search direction is defined by the formula (5). This formula acquires a vector that directs toward a direction of lowering the evaluation value. For example, xₖ may be an optimum vector acquired in the previous iteration. This Bₖ⁻¹ may be directly determined, without being based on the formula (5). It is possible that B or an inverse matrix of B is calculated based on, for example, a B formula or an H formula of the BFGS method.

By using this p, each of the plurality of arithmetic cores acquires coordinate values. For example, after p is calculated in the controller 30, different latent variables are acquired in the respective arithmetic cores.

x⁽ⁱ⁾=xₖ+aᵢpₖ (6)

The respective arithmetic cores acquire the coordinate values based on the formula (6), for example. i is the number of arithmetic core, and when, for example, n pieces of arithmetic cores acquire coordinate values in the same search direction, i is 0, 1, . . . , n. By using this i, the respective arithmetic cores acquire coordinate values, respectively, in a manner as follows, as an example.

$${x}^{( i )} = {x}_{k} + \frac{1}{{2}^{i}} {p}_{k} \qquad ( 7 )$$

For example, it is possible to set that aᵢ=2⁻ⁱ, as expressed in the formula (7). In this case, a value as a result of multiplying the search direction p by ½ is acquired in the arithmetic core 3201, and a value as a result of multiplying the search direction p by ¼ is acquired in the arithmetic core 3202, to thereby calculate the values as new latent variables. The control of such calculation is executed by the controller 30.

Note that the way of defining aᵢ is indicated as an example, and it is possible to employ, as aᵢ, an exponentiation of other than 2, for example, it is possible to set that aᵢ=(√2)⁻ⁱ. As another example, it is possible to employ, not the exponentiation, but a value as a result of equally dividing a predetermined distance from xₖ (aᵢ=i/j, in which j is set to a division number (parallel number)), simply an inverse number of i (aᵢ=1/i) or the like, or a logarithm, for example, and in addition to the above, it is also possible to use a method capable of properly performing local search.

The respective arithmetic cores calculate coordinate values based on the formula (7), and then acquire structural formulas from the respective coordinate values, thereby calculating docking scores. This can be expressed by the following formula.

f(x⁽ⁱ⁾)=f(xₖ+αᵢpₖ) (8)

Specifically, in each arithmetic core, the latent variable acquirer 10, the structural formula acquirer 12, and the score calculator 14 are implemented, and the evaluation value is calculated.

This implementation is realized by an executable file that is compiled on-line when being executed, the executable file being described by, for example, a machine language, an intermediate code (byte code), or ASCII, and the controller 30 may perform control so as to execute the implementation in each arithmetic core.

As described above, the arithmetic cores can perform the parallel arithmetic operation. For this reason, it becomes possible to execute the acquisition of a plurality of different latent variables by the metaheuristic algorithm, and the calculation of evaluation values (docking scores) in a plurality of conformations of structural formulas with respect to the respective latent variables, in a parallel manner.

For example, when the PSO, the Monte Carlo method, and the BFGS method are used as described above, a plurality of latent variables are acquired to infer a plurality of structural formulas in a parallel manner in the PSO, regarding coordinate values of the structural formulas selected by the Monte Carlo method, the BFGS method is used to calculate evaluation values f(x) at the plurality of coordinate values in a parallel manner, and the values are used as evaluation values in the Monte Carlo method. As a concrete example, a step of determining a in the formula (6) that minimizes the evaluation value f(x), can be performed as not successive calculation but parallel calculation. Accordingly, it becomes possible to perform the search of local optimum value based on coordinate values in one compound in a parallel manner, and further, by performing the search in a plurality of compounds in a parallel manner, it becomes possible to execute high-speed arithmetic processing regarding the evaluation with respect to the plurality of compounds.

For example, based on the successive calculation, a maximum arithmetic operation time is one taken for performing arithmetic operation with respect to all of a, namely, it becomes n×(1 arithmetic operation time) in the above example, and becomes n×(1 arithmetic operation time)/2 on average. On the other hand, by executing the parallel arithmetic operation as described above, it becomes possible to perform processing in (1 arithmetic operation time).

Further, even in a case where n pieces of evaluation values are calculated and then a latent variable being a minimum value is searched, by executing the parallel arithmetic operation by the plurality of arithmetic cores, it becomes possible to statistically increase the speed of operation. For example, when successively searching for the minimum value, a comparison arithmetic operation time of O(n) is required. On the other hand, even in a simple parallel arithmetic operation based on SIMD, the arithmetic operation time can be set to O(log₂ n), and depending on algorithms, by executing the parallel arithmetic operation using the plurality of arithmetic cores, it is also possible to optimize the arithmetic operation time to O(log₂ n) or less.

A difference between these arithmetic operation times becomes more significant as n becomes large. Generally, the number of coordinate values such as positions and conformations of atoms with respect to one structural formula, is not a small number. For this reason, n can be assumed to be a large number to some extent. By executing the above-described parallel arithmetic operation in such a case, it becomes possible to realize the reduction in arithmetic operation time. Further, by performing the arithmetic operation in a parallel manner, it becomes possible to realize the search in a wider range and at a higher sampling rate, resulting in that a stable and optimum solution can be acquired.

In the PSO, the processing is executed with respect to the plurality of compounds while updating velocities and positions. With the use of the inferring device 1 according to the present embodiment, the processing of the plurality of compounds is performed by the parallel arithmetic operation, to thereby realize first parallel processing. By using the metaheuristic algorithm such as the PSO, it is possible to realize the global search that avoids convergence to the local solution, in the latent space used for generating structures of compounds. For example, if particles of PSO are set to a group of m pieces, by performing an arithmetic operation for each compound, namely, by performing m-parallel arithmetic operation, the global search in the latent space is performed in a parallel manner. For example, in the PSO, by increasing the number of particles, the possibility of convergence to the local solution can be reduced.

In the arithmetic operation regarding one compound in the PSO, the Monte Carlo method is executed as in the above-described example, but the local search of the coordinate values to be a candidate for the next coordinate values in the Monte Carlo method is executed by the BFGS method. The BFGS method indicated as this example is executed by n-parallel arithmetic operation. Accordingly, it becomes possible to realize m x n-parallel arithmetic operation as a whole.

In the structural formula acquired by the PSO, since it is not known at which configuration and conformation the compound bonds to target protein, it is desirable that a space of the configuration and the conformation which the compound may have is searched over a range as wide as possible, to thereby calculate a more accurate docking score. For the search in the space of three-dimensional structure, the Monte Carlo method is used as an example.

By employing the Monte Carlo method, it is possible to realize the global search in the space of three-dimensional structure which one structural formula may have. Here, the BFGS method is used for further improving the accuracy of candidate for the solution obtained by the Monte Carlo method.

With respect to the coordinate values acquired by the Monte Carlo method, a score functional calculation is executed to calculate a score gradient. Subsequently, based on this gradient, n-parallel processing is executed by the BFGS method. By executing the BFGS method, it is possible to acquire an evaluation value with higher accuracy of a candidate for the next coordinate values by the Monte Carlo method.

From a plurality of docking scores obtained through the parallel arithmetic operation, an optimum docking score being a result of the BFGS method is acquired. Based on this docking score acquired by the BFGS method, the arithmetic operation of optimum docking score is repeatedly performed through the Monte Carlo simulation.

When a termination condition of the Monte Carlo simulation is satisfied, it is possible to acquire a docking score with high accuracy with respect to one compound. By using this result of Monte Carlo simulation, a position and a velocity with respect to the compound are updated by the PSO. This update of the position and the velocity is executed in each of the plurality of compounds, through the parallel arithmetic operation. Further, the arithmetic operation of the PSO is repeated if necessary.

In the present embodiment, by executing the m-parallel arithmetic operation by the PSO and the n-parallel arithmetic operation by the BFGS method in a combined manner, a high-speed arithmetic operation is realized.

As described above, according to the present embodiment, the inferring device can infer a proper structural formula at a higher speed in a more stabilized manner.

Note that as described above, although the present embodiment has explained the solving method using the PSO as an example of the metaheuristic algorithm, even if the metaheuristic algorithm other than the PSO is employed, it becomes possible to similarly realize the inference of a large number of structural formulas in a parallel manner.

Although it has been described above that the PSO and the BFGS method are processed in a parallel manner, as an example, the Monte Carlo method, in addition to that, may also be processed in a parallel manner. For example, in one step in the Monte Carlo simulation, the parallel processing may be performed on a plurality of coordinate values, to thereby perform the search. For instance, if this parallel processing is set to one parallel processing, the above-described processing corresponds to m×1×n-parallel processing. When the Hill Climbing method is used, for example, by executing the search through the parallel processing and comparing scores calculated by the BFGS method, it becomes possible to search for the next coordinate values at a higher speed. When the SA method is used, for example, by executing the search through the parallel processing and searching for docking scores of the respective coordinate values after being subjected to weighting by taking a temperature influence into consideration, it is possible to execute the arithmetic operation at a high speed.

The trained models of above embodiments may be, for example, a concept that includes a model that has been trained as described and then distilled by a general method.

Some or all of each device (the inference device 1) in the above embodiment may be configured in hardware, or information processing of software (program) executed by, for example, a CPU (Central Processing Unit), GPU (Graphics Processing Unit). In the case of the information processing of software, software that enables at least some of the functions of each device in the above embodiments may be stored in a non-volatile storage medium (non-volatile computer readable medium) such as CD-ROM (Compact Disc Read Only Memory) or USB (Universal Serial Bus) memory, and the information processing of software may be executed by loading the software into a computer. In addition, the software may also be downloaded through a communication network. Further, entire or a part of the software may be implemented in a circuit such as an ASIC (Application Specific Integrated Circuit) or FPGA (Field Programmable Gate Array), wherein the information processing of the software may be executed by hardware.

A storage medium to store the software may be a removable storage media such as an optical disk, or a fixed type storage medium such as a hard disk, or a memory. The storage medium may be provided inside the computer (a main storage device or an auxiliary storage device) or outside the computer.

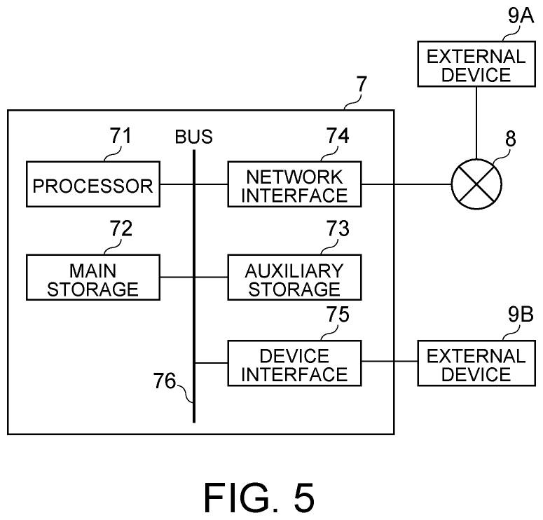

FIG. 5 is a block diagram illustrating an example of a hardware configuration of each device (the inference device 1) in the above embodiments. As an example, each device may be implemented as a computer 7 provided with a processor 71, a main storage device 72, an auxiliary storage device 73, a network interface 74, and a device interface 75, which are connected via a bus 76.

The computer 7 of FIG. 5 is provided with each component one by one but may be provided with a plurality of the same components. Although one computer 7 is illustrated in FIG. 5, the software may be installed on a plurality of computers, and each of the plurality of computer may execute the same or a different part of the software processing. In this case, it may be in a form of distributed computing where each of the computers communicates with each of the computers through, for example, the network interface 74 to execute the processing. That is, each device (the inference device 1) in the above embodiments may be configured as a system where one or more computers execute the instructions stored in one or more storages to enable functions. Each device may be configured such that the information transmitted from a terminal is processed by one or more computers provided on a cloud and results of the processing are transmitted to the terminal.

Various arithmetic operations of each device (the inference device 1) in the above embodiments may be executed in parallel processing using one or more processors or using a plurality of computers over a network. The various arithmetic operations may be allocated to a plurality of arithmetic cores in the processor and executed in parallel processing. Some or all the processes, means, or the like of the present disclosure may be implemented by at least one of the processors or the storage devices provided on a cloud that can communicate with the computer 7 via a network. Thus, each device in the above embodiments may be in a form of parallel computing by one or more computers.

The processor 71 may be an electronic circuit (such as, for example, a processor, processing circuitry, processing circuitry, CPU, GPU, FPGA, or ASIC) that executes at least controlling the computer or arithmetic calculations. The processor 71 may also be, for example, a general-purpose processing circuit, a dedicated processing circuit designed to perform specific operations, or a semiconductor device which includes both the general-purpose processing circuit and the dedicated processing circuit. Further, the processor 71 may also include, for example, an optical circuit or an arithmetic function based on quantum computing.

The processor 71 may execute an arithmetic processing based on data and/or a software input from, for example, each device of the internal configuration of the computer 7, and may output an arithmetic result and a control signal, for example, to each device. The processor 71 may control each component of the computer 7 by executing, for example, an OS (Operating System), or an application of the computer 7.

Each device (the inference device 1) in the above embodiments may be enabled by one or more processors 71. The processor 71 may refer to one or more electronic circuits located on one chip, or one or more electronic circuitries arranged on two or more chips or devices. In the case of a plurality of electronic circuitries are used, each electronic circuit may communicate by wired or wireless.

The main storage device 72 may store, for example, instructions to be executed by the processor 71 or various data, and the information stored in the main storage device 72 may be read out by the processor 71. The auxiliary storage device 73 is a storage device other than the main storage device 72. These storage devices shall mean any electronic component capable of storing electronic information and may be a semiconductor memory. The semiconductor memory may be either a volatile or non-volatile memory. The storage device for storing various data or the like in each device (the inference device 1) in the above embodiments may be enabled by the main storage device 72 or the auxiliary storage device 73 or may be implemented by a built-in memory built into the processor 71. For example, the storages 16 in the above embodiments may be implemented in the main storage device 72 or the auxiliary storage device 73.

In the case of each device (the inference device 1) in the above embodiments is configured by at least one storage device (memory) and at least one of a plurality of processors connected/coupled to/with this at least one storage device, at least one of the plurality of processors may be connected to a single storage device. Or at least one of the plurality of storages may be connected to a single processor. Or each device may include a configuration where at least one of the plurality of processors is connected to at least one of the plurality of storage devices. Further, this configuration may be implemented by a storage device and a processor included in a plurality of computers. Moreover, each device may include a configuration where a storage device is integrated with a processor (for example, a cache memory including an L1 cache or an L2 cache).

The network interface 74 is an interface for connecting to a communication network 8 by wireless or wired. The network interface 74 may be an appropriate interface such as an interface compatible with existing communication standards. With the network interface 74, information may be exchanged with an external device 9A connected via the communication network 8. Note that the communication network 8 may be, for example, configured as WAN (Wide Area Network), LAN (Local Area Network), or PAN (Personal Area Network), or a combination of thereof, and may be such that information can be exchanged between the computer 7 and the external device 9A. The internet is an example of WAN, IEEE802.11 or Ethernet (registered trademark) is an example of LAN, and Bluetooth (registered trademark) or NFC (Near Field Communication) is an example of PAN.

The device interface 75 is an interface such as, for example, a USB that directly connects to the external device 9B.

The external device 9A is a device connected to the computer 7 via a network. The external device 9B is a device directly connected to the computer 7.

The external device 9A or the external device 9B may be, as an example, an input device. The input device is, for example, a device such as a camera, a microphone, a motion capture, at least one of various sensors, a keyboard, a mouse, or a touch panel, and gives the acquired information to the computer 7. Further, it may be a device including an input unit such as a personal computer, a tablet terminal, or a smartphone, which may have an input unit, a memory, and a processor.

The external device 9A or the external device 9B may be, as an example, an output device. The output device may be, for example, a display device such as, for example, an LCD (Liquid Crystal Display), or an organic EL (Electro Luminescence) panel, or a speaker which outputs audio. Moreover, it may be a device including an output unit such as, for example, a personal computer, a tablet terminal, or a smartphone, which may have an output unit, a memory, and a processor.

Further, the external device 9A or the external device 9B may be a storage device (memory). The external device 9A may be, for example, a network storage device, and the external device 9B may be, for example, an HDD storage.

Furthermore, the external device 9A or the external device 9B may be a device that has at least one function of the configuration element of each device (the inference device 1) in the above embodiments. That is, the computer 7 may transmit a part of or all of processing results to the external device 9A or the external device 9B, or receive a part of or all of processing results from the external device 9A or the external device 9B.

In the present specification (including the claims), the representation (including similar expressions) of “at least one of a, b, and c” or “at least one of a, b, or c” includes any combinations of a, b, c, a-b, a-c, b-c, and a-b-c. It also covers combinations with multiple instances of any element such as, for example, a-a, a-b-b, or a-a-b-b-c-c. It further covers, for example, adding another element d beyond a, b, and/or c, such that a-b-c-d.

In the present specification (including the claims), the expressions such as, for example, “data as input,” “using data,” “based on data,” “according to data,” or “in accordance with data” (including similar expressions) are used, unless otherwise specified, this includes cases where data itself is used, or the cases where data is processed in some ways (for example, noise added data, normalized data, feature quantities extracted from the data, or intermediate representation of the data) are used. When it is stated that some results can be obtained “by inputting data,” “by using data,” “based on data,” “according to data,” “in accordance with data” (including similar expressions), unless otherwise specified, this may include cases where the result is obtained based only on the data, and may also include cases where the result is obtained by being affected factors, conditions, and/or states, or the like by other data than the data. When it is stated that “output/outputting data” (including similar expressions), unless otherwise specified, this also includes cases where the data itself is used as output, or the cases where the data is processed in some ways (for example, the data added noise, the data normalized, feature quantity extracted from the data, or intermediate representation of the data) is used as the output.

In the present specification (including the claims), when the terms such as “connected (connection)” and “coupled (coupling)” are used, they are intended as non-limiting terms that include any of “direct connection/coupling,” “indirect connection/coupling,” “electrically connection/coupling,” “communicatively connection/coupling,” “operatively connection/coupling,” “physically connection/coupling,” or the like. The terms should be interpreted accordingly, depending on the context in which they are used, but any forms of connection/coupling that are not intentionally or naturally excluded should be construed as included in the terms and interpreted in a non-exclusive manner.

In the present specification (including the claims), when the expression such as “A configured to B,” this may include that a physically structure of A has a configuration that can execute operation B, as well as a permanent or a temporary setting/configuration of element A is configured/set to actually execute operation B. For example, when the element A is a general-purpose processor, the processor may have a hardware configuration capable of executing the operation B and may be configured to actually execute the operation B by setting the permanent or the temporary program (instructions). Moreover, when the element A is a dedicated processor, a dedicated arithmetic circuit, or the like, a circuit structure of the processor or the like may be implemented to actually execute the operation B, irrespective of whether or not control instructions and data are actually attached thereto.

In the present specification (including the claims), when a term referring to inclusion or possession (for example, “comprising/including,” “having,” or the like) is used, it is intended as an open-ended term, including the case of inclusion or possession an object other than the object indicated by the object of the term. If the object of these terms implying inclusion or possession is an expression that does not specify a quantity or suggests a singular number (an expression with a or an article), the expression should be construed as not being limited to a specific number.

In the present specification (including the claims), although when the expression such as “one or more,” “at least one,” or the like is used in some places, and the expression that does not specify a quantity or suggests a singular number (the expression with a or an article) is used elsewhere, it is not intended that this expression means “one.” In general, the expression that does not specify a quantity or suggests a singular number (the expression with a or an as article) should be interpreted as not necessarily limited to a specific number.

In the present specification, when it is stated that a particular configuration of an example results in a particular effect (advantage/result), unless there are some other reasons, it should be understood that the effect is also obtained for one or more other embodiments having the configuration. However, it should be understood that the presence or absence of such an effect generally depends on various factors, conditions, and/or states, etc., and that such an effect is not always achieved by the configuration. The effect is merely achieved by the configuration in the embodiments when various factors, conditions, and/or states, etc., are met, but the effect is not always obtained in the claimed invention that defines the configuration or a similar configuration.

In the present specification (including the claims), when the term such as “maximize/maximization” is used, this includes finding a global maximum value, finding an approximate value of the global maximum value, finding a local maximum value, and finding an approximate value of the local maximum value, should be interpreted as appropriate accordingly depending on the context in which the term is used. It also includes finding on the approximated value of these maximum values probabilistically or heuristically. Similarly, when the term such as “minimize” is used, this includes finding a global minimum value, finding an approximated value of the global minimum value, finding a local minimum value, and finding an approximated value of the local minimum value, and should be interpreted as appropriate accordingly depending on the context in which the term is used. It also includes finding the approximated value of these minimum values probabilistically or heuristically. Similarly, when the term such as “optimize” is used, this includes finding a global optimum value, finding an approximated value of the global optimum value, finding a local optimum value, and finding an approximated value of the local optimum value, and should be interpreted as appropriate accordingly depending on the context in which the term is used. It also includes finding the approximated value of these optimal values probabilistically or heuristically.

In the present specification (including claims), when a plurality of hardware performs a predetermined process, the respective hardware may cooperate to perform the predetermined process, or some hardware may perform all the predetermined process. Further, a part of the hardware may perform a part of the predetermined process, and the other hardware may perform the rest of the predetermined process. In the present specification (including claims), when an expression (including similar expressions) such as “one or more hardware perform a first process and the one or more hardware perform a second process,” or the like, is used, the hardware that perform the first process and the hardware that perform the second process may be the same hardware, or may be the different hardware. That is: the hardware that perform the first process and the hardware that perform the second process may be included in the one or more hardware. Note that, the hardware may include an electronic circuit, a device including the electronic circuit, or the like.

In the present specification (including the claims), when a plurality of storage devices (memories) store data, an individual storage device among the plurality of storage devices may store only a part of the data or may store the entire data. Further, some storage devices among the plurality of storage devices may include a configuration for storing data.

While certain embodiments of the present disclosure have been described in detail above, the present disclosure is not limited to the individual embodiments described above. Various additions, changes, substitutions, partial deletions, etc. are possible to the extent that they do not deviate from the conceptual idea and purpose of the present disclosure derived from the contents specified in the claims and their equivalents. For example, when numerical values or mathematical formulas are used in the description in the above-described embodiments, they are shown for illustrative purposes only and do not limit the scope of the present disclosure. Further, the order of each operation shown in the embodiments is also an example, and does not limit the scope of the present disclosure.

1. An inferring device comprising:
one or more memories; and
one or more processors configured to:
acquire a plurality of latent variables;
generate a plurality of structural formulas by inputting the plurality of latent variables, respectively, into a model; and
calculate a plurality of scores by evaluating the plurality of structural formulas, respectively, wherein:
the one or more processors execute processing of the acquisition of the plurality of latent variables, the generation of the plurality of structural formulas, and the calculation of the plurality of scores, at least two times or more; and
the one or more processors acquire, based on the acquired plurality of latent variables and the calculated plurality of scores, the plurality of latent variables in any of the execution of at least second time or thereafter.

2. The inferring device according to claim 1, wherein
the one or more processors acquire the plurality of latent variables based on a metaheuristic algorithm.

3. The inferring device according to claim 2, wherein
the metaheuristic algorithm is at least one of a particle swarm optimization, an artificial bee colony method, a simulated annealing method, a Metropolis method, a Monte Carlo method, a hill climbing method, an ant colony optimization method, a harmony search, a cuckoo search, a spiral optimization method, a firefly algorithm, a genetic algorithm, an immune algorithm, a covariance matrix adaptation evolution strategy, an evolution strategy, or an amoeba method/Nelder-Mead method.

4. The inferring device according to claim 1, wherein
the one or more processors execute at least any processing of the acquisition of the plurality of latent variables, the generation of the plurality of structural formulas, or the calculation of the plurality of scores, in a parallel manner.

5. The inferring device according to claim 4, wherein
the one or more processors acquire the plurality of latent variables based on a metaheuristic algorithm.

6. The inferring device according to claim 1, wherein
the one or more processors calculate the score based on a three-dimensional structure of a compound expressed by the structural formula.

7. The inferring device according to claim 6, wherein
the one or more processors calculate the score by performing a simulation of a docking of the compound.

8. The inferring device according to claim 7, wherein
the one or more processors calculate the score based on a potential.

9. The inferring device according to claim 7, wherein
the one or more processors calculate the score based on at least any of a docking position, a docking direction, or internal coordinates.

10. The inferring device according to claim 7, wherein
the one or more processors execute the simulation of the docking by using a first method which executes a global search, and a second method which executes a search in a more local manner than the first method.

11. The inferring device according to claim 10, wherein
the one or more processors execute at least the search using the first method in a parallel manner.

12. The inferring device according to claim 1, wherein
the one or more processors calculate the score based on at least one of a three-dimensional structure, an avoidance structure, or an easiness of bonding with respect to predetermined protein, of a compound expressed by the structural formula.

13. The inferring device according to claim 1, wherein
the score is determined based on a plurality of properties.

14. The inferring device according to claim 1, wherein
the one or more processors calculate a plurality of kinds of the score.

15. The inferring device according to claim 1, wherein
the score is an evaluation value based on a property of a compound expressed by the structural formula.

16. The inferring device according to claim 1, wherein
the structural formula is information indicating at least either a molecular structure or a crystal structure.

17. The inferring device according to claim 1, wherein
the structural formula is information expressed by a graph.

18. The inferring device according to claim 1, wherein
the acquisition of the plurality of latent variables in the execution of the first time is for acquiring initial values of the plurality of latent variables.

19. An inferring method comprising:
making one or more processors acquire a plurality of latent variables;
making the one or more processors generate a plurality of structural formulas by inputting the plurality of latent variables, respectively, into a model; and
making the one or more processors calculate a plurality of scores by evaluating the plurality of structural formulas, respectively, wherein:
the one or more processors execute processing of the acquisition of the plurality of latent variables, the generation of the plurality of structural formulas, and the calculation of the plurality of scores, at least two times or more; and
the one or more processors acquire, based on the acquired plurality of latent variables and the calculated plurality of scores, the plurality of latent variables in any of the execution of at least second time or thereafter.

20. A non-transitory computer readable medium storing a program, the program configured to:
making one or more processors acquire a plurality of latent variables;
making the one or more processors generate a plurality of structural formulas by inputting the plurality of latent variables, respectively, into a model; and
making the one or more processors calculate a plurality of scores by evaluating the plurality of structural formulas, respectively, wherein:
the one or more processors are made to execute processing of the acquisition of the plurality of latent variables, the generation of the plurality of structural formulas, and the calculation of the plurality of scores, at least two times or more; and
the one or more processors are made to acquire, based on the acquired plurality of latent variables and the calculated plurality of scores, the plurality of latent variables in any of the execution of at least second time or thereafter.
